(19) United States
(12) Patent Application Publication
(10) Pub. No.: US 2007/0049763 A1
(43) Pub. Date: Mar. 1, 2007

(54) Methods for preparation and use of strong base catalysts

(76) Inventors: Martin J. Reaney, Saskatoon (CA); Neil D. Westcott, Saskatoon (CA)

Correspondence Address: Ralph A. Dowell of DOWELL & DOWELL P.C., 2111 Eisenhower Ave, Suite 406, Alexandria, VA, 22314, US

(21) Appl. No.: 11/208,730

(22) Filed: Aug. 23, 2005

(51) Int. Cl. C07C 51/347

(52) U.S. Cl. 554/126

(57) ABSTRACT

Methods for preparation of a unique superbase catalyst consisting of mixture of polyether alcohol and base in which a polyether alcohol superbase is produced by the removal of water or alcohol at elevated temperatures to form a polyether alcohol alkoxide. The superbase catalyst is useful in, but not limited to, quantitative isomerization of alkyl esters of vegetable oils containing interrupted double bond systems to yield esters with conjugated double bond systems.

### FIELD OF INVENTION

This invention relates to a process for preparation and application of a novel strong base catalyst. The strong base is useful in conversion of conjugated linoleic acid (CLA) from alkyl esters of C1-C5 alkanols derived from oils rich in linoleic acid and conjugated linolenic acids from alkyl esters of C1-C5 alkanols derived from oils rich in linolenic acid. The reaction with alkyl esters of linoleic acid produces approximately equal amounts of the CLA isomers 9Z,11E-octadecadienoic acid and 10E,12Z-octadecadienoic acid. The reaction with alfa-linolenic acid produces a mixture of 9,13,15 Z,E,Z-octadecatrienoic acid, 9,11,15-Z,E,Z-octadecatrienoic acid and 10,12,14-E,Z,E-octadecatrienoic acid The reaction is unique in the reaction proceeds rapidly at temperatures as low as 20° C. and requires only catalytic amounts of the strong base and polyether alcohol.

### BACKGROUND OF THE INVENTION

In synthetic organic chemistry base catalysts may be divided into classes of base strength. Depending on the base strength different catalyzed reactions are possible with each class of base. Metal carbonates and hydroxides such as sodium and potassium hydroxide are efficient catalysts for transesterification and have been used to produce sucrose polyesters and alkyl esters. Strong base catalysts such as metal alkoxides (egs. Sodium methylate, potassium tertiary butryate) are broadly used in commercial organic syntheses and often preferred in specific reactions. The strong bases are often capable of catalyzing reactions at lower temperatures and in less expensive solvent systems. While some of these bases are prone to oxidation all are prone to inactivation by reaction with water.

Conjugated linoleic acid is the trivial name given to a series of eighteen carbon diene fatty acids with conjugated double bonds. Applications of conjugated linoleic acids vary from treatment of medical conditions such as anorexia (U.S. Pat. No. 5,430,066) and low immunity (U.S. Pat. No. 5,674,901) to applications in the field of dietetics where CLA has been reported to reduce body fat (U.S. Pat. No. 5,554,646) and to inclusion in cosmetic formulae (U.S. Pat. No. 4,393,043). CLA shows similar activity in veterinary applications. In addition, CLA has proven effective in reducing valgus and varus deformity in poultry (U.S. Pat. No. 5,760,083), and attenuating allergic responses (U.S. Pat. No. 5,585,400). CLA has also been reported to increase feed conversion efficiency in animals (U.S. Pat. No. 5,428,072). CLA-containing bait can reduce the fertility of scavenger bird species such as crows and magpies (U.S. Pat. No. 5,504,114).

Industrial applications for CLA also exist where it is used as a lubricant constituent (U.S. Pat. No. 4,376,711). CLA synthesis can be used as a means to chemically modify linoleic acid so that it is readily reactive to Diels-Alder reagents (U.S. Pat. No. 5,053,534). In one method linoleic acid was separated from oleic acid by first conjugation then reaction with maleic anhydride followed by distillation (U.S. Pat. No. 5,194,640).

Conjugated linoleic acid occurs naturally in ruminant depot fats. The predominant form of CLA in ruminant fat is the 9Z,11E-octadecadienoic acid which is synthesized from linoleic acid in the rumen by micro-organisms like Butryvibrio fibrisolvens. The level of CLA found in ruminant fat is in part a function of dietary 9Z,12Z-octadecadienoic acid and the level of CLA in ruminant milk and depot fat may be increased marginally by feeding linoleic acid (U.S. Pat. No. 5,770,247).

CLA may also be prepared by any of several analytical and preparative methods. Pariza and Ha pasteurized a mixture of butter oil and whey protein at 85° C. for 5 minutes and noted elevated levels of CLA in the oil (U.S. Pat. No. 5,070,104). CLA produced by this mechanism is predominantly a mixture of 9Z,11E-octadecadienoic acid and 10E,12Z-octadecadienoic acid. CLA has also been produced by the reaction of soaps with strong alkali bases in molten soaps, alcohol, and ethylene glycol monomethyl ether (U.S. Pat. Nos. 2,389,260; 2,242,230 & 2,343,644). These reactions are inefficient, as they require the multiple steps of formation of the fatty acid followed by production of soap from the fatty acids, and subsequently increasing the temperature to isomerize the linoleic soap. The CLA product is generated by acidification with a strong acid (sulfuric or hydrochloric acid) and repeatedly washing the product with brine or CaCl₂.

Iwata et al. (U.S. Pat. No. 5,986,116) overcame the need for an intermediate step of preparation of fatty acids by reacting oils directly with alkali catalyst in a solvent of propylene glycol under low water or anhydrous conditions. Reaney et al. (Reaney, Liu and Westcott (1999) Commercial production of CLA. In Yurawecz, Mossaba, Kramer, Pariza and Nelson Eds. Advances in conjugated linoleic acid research, Vol. 1 pp.) identified that CLA products prepared in the presence of glycol and other alcohols may transesterify with the glycerol and produce esters of the glycol. Such esters have been identified by Reaney (unpublished work) in commercial products and in CLA prepared in propylene glycol by the method of U.S. Pat. No. 5,986,116. The biological activity of esters of CLA containing fatty acids and propylene glycol is relatively high and therefore their presence in the CLA product is undesirable.

CLA has been synthesized from fatty acids using SO₂ in the presence of a sub-stoichiometric amount of soap forming base (U.S. Pat. No. 4,381,264). The reaction with this catalyst produced predominantly the all trans configuration of CLA.

Baltes, Wechmann and Weghorst (U.S. Pat. No. 3,162,658) achieved the conjugation of distilled methyl esters of soybean oil by the addition of 10 percent potassium methylate at 120° C. in five hours. The reaction produced 97% conjugation of the available double bonds.

Ritz and Reese (U.S. Pat. No. 3,984,444) found that aprotic solvents were suitable for the formation of conjugated bonds in soybean oil. They report mixing 500 g of soy oil with 500 g of DMSO at 50° C. and then adding 5 grams of finely divided potassium methylate. The reaction produced 97% conjugation of the available double bonds

Efficient synthesis of 9Z,11E-octadecadienoic from ricinoleic acid has been achieved (Russian Patent 2,021,252). This synthesis, although efficient, uses expensive elimination reagents such as 1,8-diazobicyclo-(5,4,0)-undecene. For most applications the cost of the elimination reagent increases the production cost beyond the level at which commercial production of CLA is economically viable.

Of these methods alkali isomerization of soaps is the least expensive process for bulk preparation of CLA isomers, however, the use of either monohydric or polyhydric alcohols in alkali isomerization of CLA can be problematic. Lower alcohols are readily removed from the CLA product but they require the production facility be built to support the use of flammable solvents. Higher molecular weight alcohols and polyhydric alcohols are considerably more difficult to remove from the product and residual levels of these alcohols (e.g. ethylene glycol) may not be acceptable in the CLA product.

Water may be used in place of alcohols in the production of CLA by alkali isomerization of soaps (U.S. Pat. Nos. 2,350,583 and 4,164,505). When water is used for this reaction it is necessary to perform the reaction in a pressure vessel whether in a batch (U.S. Pat. No. 2,350,583) or continuous mode of operation (U.S. Pat. No. 4,164,505). The process for synthesis of CLA from soaps dissolved in water still requires a complex series of reaction steps. Bradley and Richardson (Industrial and Engineering Chemistry February 1942 vol 34 no 2 237-242) were able to produce CLA directly from soybean triglycerides by mixing sodium hydroxide, water and oil in a pressure vessel. Their method eliminated the need to synthesize fatty acids and then form soaps prior to the isomerization reaction. However, they reported that they were able to produce oil with up to 40 percent CLA. Quantitative conversion of the linoleic acid in soybean oil to CLA would have produced a fatty acid mixture with approximately 54 percent CLA.

In order to overcome the high cost of alkali and solvent often encountered in CLA production Reaney (U.S. Pat. No. 6,409,649) developed a method for utilizing the waste alkaline glycerol from biodiesel synthesis as a catalyst and medium for CLA production. Similarly Reaney (U.S. Pat. No. 6,414,171) describe the direct conversion of soapstock from the alkaline treatment of vegetable oils to CLA. This conversion has the advantage of using water as the reaction medium and the presence of large amounts of alkali in the soap. Though inexpensive, both reactions require heating the reaction mixture to temperatures above 190° C.

Commercial conjugated linoleic acid often contains a mixture of positional isomers that may include 8E,10Z-octadecadienoic acid, 9Z,11E-octadecadienoic acid, 10E,12Z-octadecadienoic acid, and 11Z,13E-octadecadienoic acid (Christie, W. W., G. Dobson, and F. D. Gunstone, (1997) Isomers in commercial samples of conjugated linoleic acid. J. Am. Oil Chem. Soc. 74, 11, 1231).

The present invention describes a method of production of CLA using polyethylene glycol alone or with a co-solvent as a reaction medium and a vegetable oil containing more than 60% linoleic acid. The reaction products in polyether glycol containing solvent are primarily 9Z,11E-octadecadienoic acid and 10E,12Z-octadecadienoic acid in equal amounts. The reaction product is readily released by acidification.

### SUMMARY OF THE INVENTION

In the present invention a strong base solution is prepared which is suitable for catalyzing numerous reactions. The strong base is produced by the mixture of simple commercially available starting materials including both alkali hydroxide base and a polyether alcohol solvent. When this mixture is heated under vacuum a reaction takes place wherein water is released and viscosity rises. Surprisingly the product of this reaction is an unusually powerful base that has advantageous properties in chemical synthesis using base catalyst. The strong base is non-volatile and non-toxic. It has greater potency than many conventional strong base solutions as the ether alcohol solvents act as a phase transfer solvent to assist in the reaction.

Thus, by one aspect of the invention there is provided a process for producing a polyethylene alkylate catalyst comprising reacting an alkali base, selected from the group consisting of hydroxide, alkoxide, metal and hydride, with a polyether alcohol solvent, under vacuum at a temperature in the range of 100° C.-150° C., so as to produce a non volatile, non toxic polyether alkylate catalyst.

By another aspect of this invention there is provided a strong base catalyst composition comprising a non volatile, non toxic polyether alkylate produced by reaction between an alkali base, selected from the group consisting of hydroxide, alkoxide, metal and hydride, and polyether alcohol.

By yet another aspect of this invention there is provided a process for producing an isomeric conjugated linoleic acid (CLA)-rich alkyl ester mixture comprising reacting a linoleic acid-rich oil in the presence of a catalytic amount of a strong base comprising a non volatile non toxic polyether alkylate at a temperature above 50° C. and separating said CLA-rich alkyl ester mixture.

### BRIEF DESCRIPTION OF DRAWINGS

FIG. 1 is a sketch illustrating the reaction of metal hydroxides with polyethylene glycol (238 grams per mole) with the release of one water molecule.

FIG. 2 is a sketch illustrating the reaction of metal ethoxides with polyethylene glycol (238 grams per mole) with the release of one ethanol molecule.

FIG. 3 is a sketch illustrating the reaction of metal with polyethylene glycol (238 grams per mole) with the release of hydrogen.

FIG. 4 is a sketch illustrating production of a preferred polyether alcohol that may generate a tertiary base.

FIG. 5 (a) is a gas chromatogram of sunflower oil methyl esters; FIG. 5 (b) is a gas chromatogram of sunflower oil methyl esters reacted according to example 11; FIG. 5(c) is a gas chromatogram of sunflower oil methyl esters reacted according to counter example 13.

FIG. 6(a) is an IR spectrum of PEG 300; and FIG. 6(b) is an IR spectrum of PEG 300 after formation of strong base catalyst as described in example 1.

FIG. 7(a) is an NMR spectrum of PEG 300; FIG. 6(b) is an NMR spectrum of PEG 300 after formation of strong base catalyst as described in example 1.

FIG. 8 is a bar graph showing materials consumed in production of CLA using (a) the catalyst used in Reaney et al. (U.S. Pat. No. 6,822,104) (Example 13) and (b) the catalyst according to the present invention (Example 11).

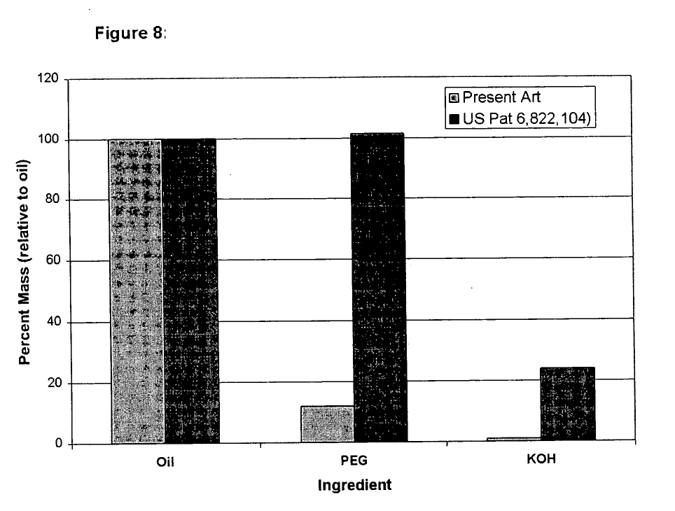

### DETAILED DESCRIPTION OF THE INVENTION

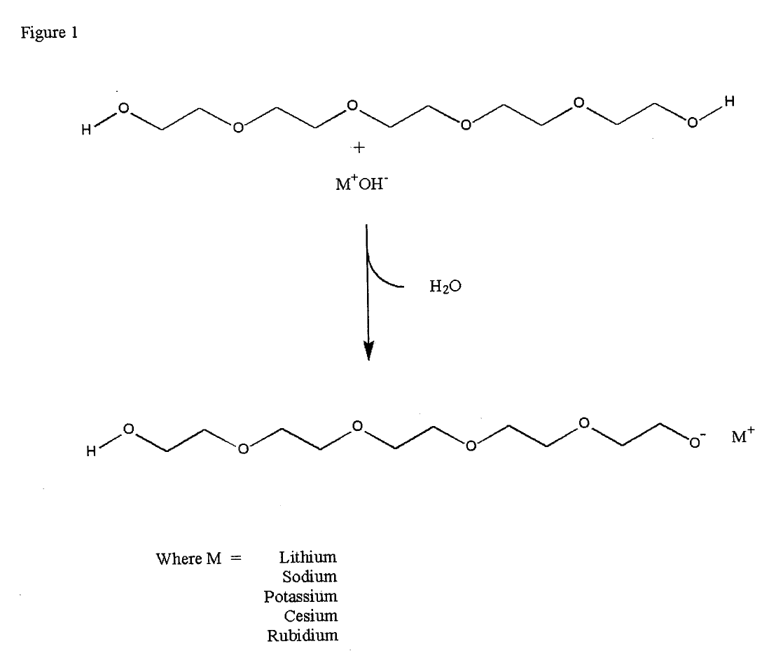

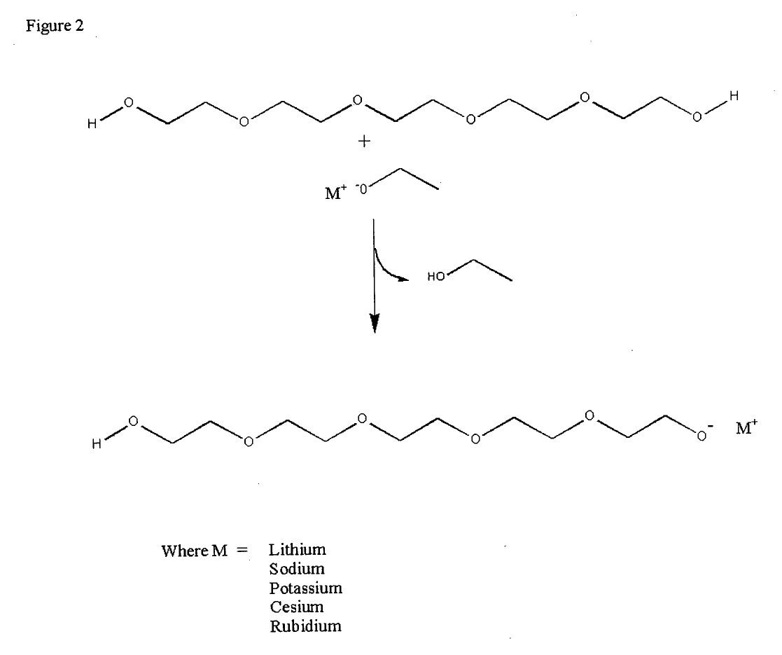

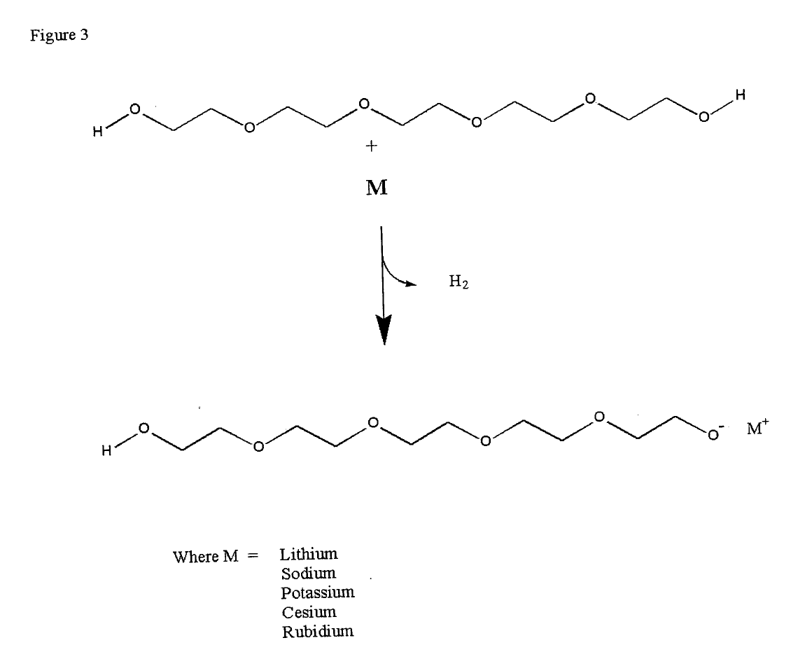

In the current art a strong base catalyst is produced by the reaction of a weaker base with a polyether alcohol using the art of the present invention to greatly increase the activity of the base. In a preferred process the base of the current invention is prepared by dissolving an amount of alkali hydroxide of a Group I alkali earth metal in the polyether alcohol and then heating the mixture under vacuum (FIG. 1). One skilled in the art would recognize that the same end product could result from a number of other potential process steps (FIGS. 2,3). For example, addition of the Group I alkali metal directly to poly ether alcohol would liberate hydrogen and result in the same product base material (FIG. 3). Although this process is less desirable due to the production of explosive hydrogen and reactive metals it is a part of the current art. The catalyst may also be produced by the reaction of alkoxides derived by reaction of Group I alkali metals with lower alkanols (FIG. 2). The alkoxides produce catalyst of the same efficacy but again they are highly sensitive to inactivation by water.

The polyether alcohol is chosen because of its low toxicity, its stability during storage and its ready ability to form an alkoxide by reaction with base. Once formed the polyether alcohol base can be used in a number of reactions to displace alkoxides of the lower alcohols in similar applications.

Formation of the catalyst may be determined by the loss of water, alcohol or hydrogen depending on the source of base used in catalyst synthesis. The accurate measurement mass loss during the synthesis can indicate the formation of the catalyst. The production of the catalyst increases the viscosity of the catalyst solution in polyether alcohol. Furthermore, the catalyst can be identified by changes both the IR and NMR spectrum of the solution. Using combined analytical methods it may be shown that the catalyst produced by reaction of aqueous alkali hydroxide solution, solid alkali hydroxide, alkoxide of lower alcohol and metal were equivalent in chemical composition.

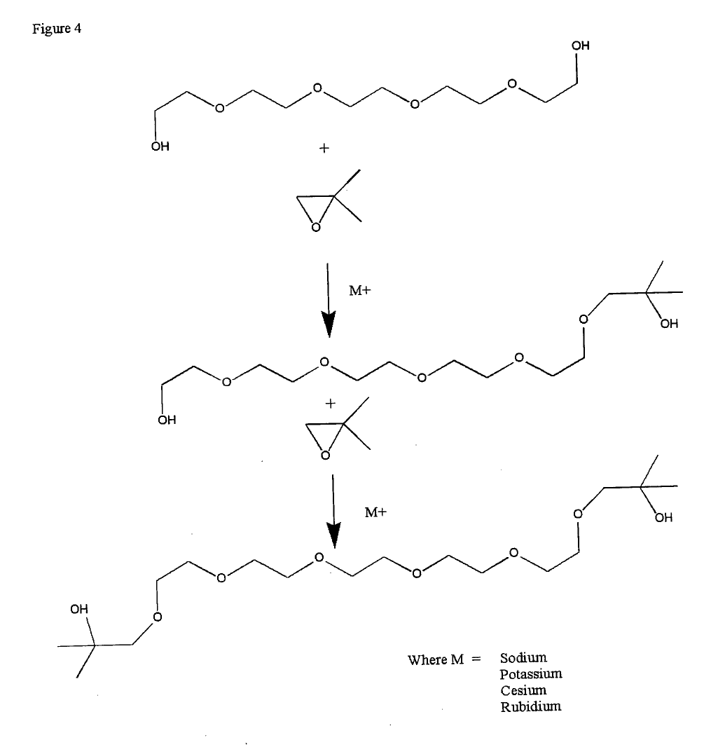

It is known by those skilled in the art that the strength of alkoxide catalysts may be affected by the nature of the alcohol. It is known, for example, that primary alcohols such as ethanol form weaker base than do tertiary alcohols like tertiary butanol. The current art includes bases made from polyether alcohols that contain primary, secondary and tertiary alcohols. FIG. 4 depicts the synthesis of a polyether alcohol that contains a tertiary alcohol group.

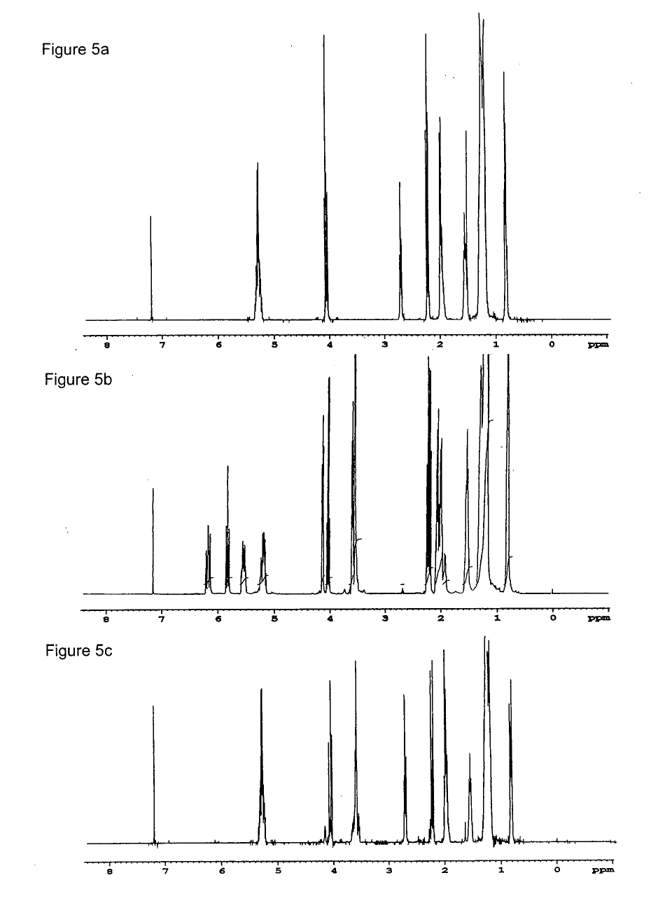

The catalyst is also characterized by its unique ability to facilitate difficult chemical reactions under mild conditions. In a preferred reaction the catalyst was utilized to conjugate the fatty alkyl esters of a linoleic acid rich oil to form conjugated linoleic acid. The conditions of this reaction are mild and produce and advantageous isomer mixtures. Reaction progress in determining the efficacy of the catalyst was determined by gas liquid chromatography and NMR spectroscopy. FIG. 5A is the chromatogram of alkyl methyl esters produced from sunflower oil. FIG. 5B is chromatogram of the product of reaction of sunflower ethyl esters according to example 11 and FIG. 5C is a chromatogram of the reaction of sunflower methyl esters according to counter example 13. As may be concluded from FIG. 5 the reaction in the current art produces primarily the preferred 9Z,11E-octadecadienoic acid and 10E,12Z-octadecadienoic acid isomeric mixture leaving little unreacted material and little of the trans, trans CLA isomer.

### EXAMPLES

### Example 1

### Preparation of Strong Base Catalyst from Peg 300 and Metal Hydroxides

Hydroxides of lithium, sodium, potassium, rubidium (solution) and cesium (monohydrate) were placed in round bottom flasks and heated to 110° C. in a vacuum oven under vacuum (29″) for 1 hour. With the exception of the rubidium hydroxide in solution there was no appreciable weight change. The rubidium solution lost a small amount of water. The color of the hydroxides remained constant with the treatment. Similarly polyethylene glycol 300 MW was placed in a round bottomed flask at the same time under vacuum. The peg solution remained clear and colorless throughout the treatment. The flasks were then removed from the heat and vacuum sources and the weight of the flask recorded. There was no change in weight of the solution. The infrared spectrum of the PEG and the PEG alkylates were recorded on samples placed between KBr salt blocks both before and after the vacuum treatment. The NMR spectra of the PEG and the PEG alkylates were recorded on samples both before and after treatment. The spectra of the untreated and treated materials were highly similar. Vacuum treatment alone did not change the composition of the PEG solution.

To each flask containing a metal hydroxide was added 10 times the weight of PEG 300. The flasks were placed in the vacuum oven at room temperature and the temperature was raised slowly to 110° C. All of the solutions boiled vigorously under the heat and vacuum treatment. All of the solutions turned to amber and then to dark brown. After vacuum treatment for 18 hours most boiling had ceased and no residual solid catalyst was present in the solutions of KOH, rubidium and cesium. Significant amounts of undissolved sodium catalyst remained in the bottom of the flask. The weight of each flask was recorded after the vacuum treatment. The FT-IR spectra of the basic solutions prepared under treatment with heat and vacuum were recorded by placing the samples between salt blocks. It was observed that each sample lost weight as would be consistent with the formation of an alkali metal alkoxide of the polyethylene glycol. The vacuum treatment substantially increased the viscosity of the PEG solution as well.

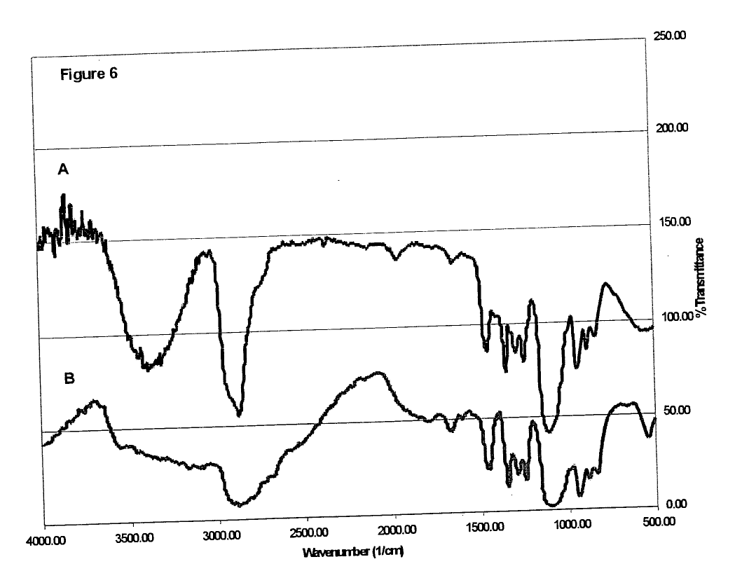

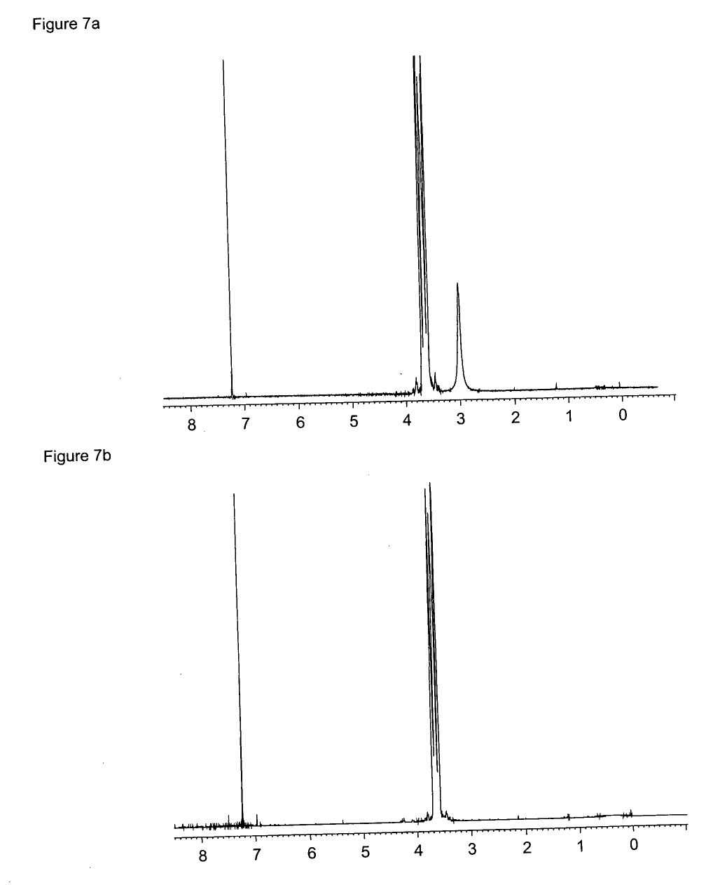

The FT-IR showed significant changes in peak absorbance. The primary difference was the lessening or disappearance of the hydroxyl absorbance at 3364 cm⁻¹ (FIG. 6) Most other peaks were unaffected but due to light scattering there was some degradation of the baseline. The NMR spectra of PEG 300 revealed a complex peak at 3.63 ppm (area=10) and a broad singlet at 2.9 ppm (area=1; FIG. 7). PEG 300 is a mixture of isomers with an average molecular weight of 300 grams per mole. The expected area ratio of peaks at 3.63 to 2.9 ppm is 13:1. This indicates that the PEG 300 signal is as it is expected. However, the NMR spectra of solutions of metal hydroxides indicated that the singlet at 2.9 ppm had disappeared.

Taken as a whole the weight loss on reaction and the disappearance of the IR and NMR peaks at 3364 cm⁻¹ and 2.9 ppm respectively are consistent with the formation of PEG alkylate.

### Example 2

### Preparation of Strong Base Catalyst from Peg 300 and Aqueous Solutions of Metal Hydroxides

Two grams of a solution of 45% potassium hydroxide in water or two grams of a solution of 50% sodium hydroxide in water were added to 13 grams of polyethylene glycol 300 in a pre-weighed round bottom flask containing a Teflon coated stirring bar. The flask was equipped with a vacuum adaptor and heated to 130° C. under vacuum (0.01 mm Hg) with stirring until all bubbling ceased. The flask was then removed from the heat and vacuum sources and the weight of the flask recorded. The FT-IR spectra of the basic solutions were recorded by placing the samples between KBr windows.

Weight loss was recorded for PEG and each base separately and the weight loss of the reactants together was also determined. Weight loss of greater than the sum of the loss of the two separate ingredients was assumed to be due to formation of the strong base PEG alkylate catalyst with the concomitant loss of water. FT-IR showed a decrease in the characteristic OH stretch absorbance of PEG solutions observed at 3365 cm⁻¹.

### Example 3

### Strong Base Catalyst is not Produced by Reaction of Peg 300 and Potassium Carbonate

Either 0.95 g of sodium carbonate or 1.41 g of potassium carbonate were added to 13 grams of polyethylene glycol 300 in a pre-weighed round bottom flask containing Teflon coated stirring bar. The flask was equipped with a vacuum adaptor and heated to 130° C. under vacuum (0.01 mm Hg) until all bubbling ceased. The flask was then removed from the heat and vacuum sources and the weight of the flask recorded. The FT-IR spectra of the basic solutions were recorded by placing the samples between KBr windows.

Weight loss was recorded for PEG and each base separately and the weight loss of the reactants together was also determined. Weight loss was minor and it was assumed that the strong base metal alkylate catalyst did not form. FT-IR showed a no decrease in the characteristic OH stretch absorbance of PEG solutions at 3365 cm⁻¹.

### Example 4

### Preparation of Strong Base Catalyst from Peg 300 and Potassium Ethoxide

One gram of freshly prepared potassium ethoxide was added to 13 grams of polyethylene glycol 300 in a pre-weighed round bottom flask containing a Teflon coated stirring bar. The flask was equipped with a vacuum adaptor and heated to 130° C. under vacuum (0.01 mm Hg) until all bubbling ceased. The flask was then removed from the heat and vacuum sources and the weight of the flask recorded. The FT-IR spectra of the basic solutions were recorded by placing the samples between KBr windows.

Weight loss was recorded for PEG and potassium ethoxide separately and the weight loss of the reactants together was also determined. Weight loss of greater than the sum of the loss of the two separate ingredients was assumed to be due to formation of the PEG alkylate strong base catalyst with the concomitant loss of alcohol. FT-IR showed a similar decrease in the characteristic OH stretch absorbance of PEG solutions at 3365 cm⁻¹ consistent with the formation of the catalyst.

### Example 5

### Preparation of Strong Base Catalyst from Peg 300 and Metal

Polyethylene glycol 300 (13 g) was added to a pre-weighed round bottom flask containing a Teflon coated stirring bar. The flask was equipped with a vacuum adaptor and heated to 130° C. under vacuum (0.01 mm Hg) until all bubbling ceased. The flask was then removed from the heat and vacuum sources and the weight of the flask recorded. Subsequently either 0.41 g of sodium or 0.70 g of potassium was added to the dry PEG. The FT-IR spectra of the basic solutions were recorded by placing the samples between KBr windows.

Weight loss was recorded for PEG and each base separately and the weight loss of the reactants together was also determined. Weight loss of greater than the sum of the loss of the two separate ingredients was assumed to be due to formation of the strong base catalyst with the concomitant loss of hydrogen. FT-IR showed a similar decrease in the characteristic OH stretch absorbance of PEG solutions at 3365 cm⁻¹ consistent with the formation of the catalyst.

### Example 6

### Preparation of Strong Base Catalyst from Calcium Hydroxide and Potassium Carbonate

Potassium carbonate (1.41 g) and calcium hydroxide (0.66 g) were added to 13 grams of polyethylene glycol 300 in a preweighed round bottom flask containing a teflon coated stirring bar. The flask was equipped with a vacuum adaptor and heated to 130° C. under vacuum (0.01 mm Hg) until all bubbling ceased. The flask was then removed from the heat and vacuum sources and the weight of the flask recorded. The FT-IR spectra of the basic solutions were recorded by placing the samples between KBr windows.

Weight loss was recorded for PEG and each base separately and the weight loss of the reactants together was also determined. Weight loss of greater than the sum of the loss of the two separate ingredients was assumed to be due to formation of the strong base catalyst with the concomitant loss of alcohol. FT-IR showed a similar decrease in the characteristic OH stretch absorbance of PEG solutions at 3365 cm⁻¹.

### Example 7

### Preparation of Strong Base Catalyst from Polyether Alcohols and Metal Hydroxides

Potassium hydroxide (1.0 g) was added to 13 grams of each of several polyether alcohols in a preweighed round bottom flask containing a teflon coated stirring bar. The polyether alcohols included PEG 200, 300, 1500, 3000, Brij 92, Brij 72 and polypropylene glycol. The flask was equipped with a vacuum adaptor and heated to 130° C. under vacuum (0.01 mm Hg) until all bubbling ceased. The flask was then removed from the evaporator and the weight of the flask recorded. The FT-IR spectra of the basic solutions were recorded by placing the samples between KBr windows.

Weight loss was recorded for each polyether alcohol and each base separately and the weight loss of the reactants together was also determined. Weight loss of greater than the sum of the loss of the two separate ingredients was assumed to be due to formation of the strong base catalyst with the concomitant loss of water. FT-IR showed a similar decrease in the characteristic OH stretch absorbance of solutions between at 3365 cm⁻.

### Example 8

### Preparation of Safflower Oil Methyl Esters with Potassium Hydroxide

Methyl esters were prepared for other examples of strong base isomerization. Methyl ester of safflower oil was prepared by alkali catalyzed alcoholysis with methanol. The base alcohol catalysis solution was prepared by mixing 200 grams of methanol with 10 grams of potassium hydroxide in a covered glass beaker. Mixing of the solid hydroxide was facilitated by adding a Teflon coated magnet and placing the beaker on a stirrer hot plate. Once the mixture was dissolved 120 grams of the solution was transferred to 1000 grams of safflower oil. This mixture was agitated for 1 hour at room temperature using a Teflon coated bar magnet on a stirrer hotplate. After 1 hour the contents of the reaction vessel were transferred to a 2 liter glass separatory funnel and allowed to separate for 4 hours. After 4 hours the lower layer containing primarily glycerin was drained and set aside the upper layer was returned to a beaker for a second stage of reaction.

The second stage of reaction was accomplished by adding the remaining catalyst alcohol solution (90 g) to the safflower oil and agitating with a Teflon stirring bar as described above for 1 hour. The reaction contents were transferred to a 2 liter glass separatory funnel and allowed to separate overnight. After settling the lower layer containing glycerin, potassium hydroxide and alcohol was removed. The upper layer was placed on a rotary evaporator to substantially remove all remaining methanol. After the alcohol was removed the methyl ester was filtered on a glass fiber filter to remove residual glycerol catalyst and soaps. The residual material was used as a safflower oil methyl ester substrate in further reactions.

### Example 9

### Preparation of Safflower Oil Ethyl Esters with Potassium Hydroxide

Ethyl ester of safflower oil was prepared by alkali catalyzed alcoholysis with ethanol. The base alcohol catalysis solution was prepared by mixing 350 grams of ethanol with 10 grams of potassium hydroxide in a covered glass beaker. Mixing of the solid hydroxide was facilitated by adding a Teflon coated magnet and placing the beaker on a stirrer hot plate. Once the mixture was dissolved it was transferred to 1000 grams of flax oil. This mixture was agitated for 2 hours at room temperature using a Teflon coated bar magnet on a stirrer hotplate. After 2 hours the contents of the reaction vessel were transferred to a 2 liter glass separatory funnel and allowed to separate for 4 hours. The lower layer containing glycerin unreacted ethanol and potassium hydroxide was removed. The upper layer was placed on a rotary evaporator to substantially remove all remaining ethanol. After the alcohol was removed the ethyl ester was filtered on a glass fiber filter to remove residual glycerol, catalyst and soaps. The residual material was used as a safflower oil ethyl ester substrate in further reactions.

### Example 10

### Preparation of Flax Oil Methyl Esters with Potassium Hydroxide

Methyl ester of flax oil was prepared by alkali catalyzed alcoholysis with methanol. The base alcohol catalysis solution was prepared by mixing 200 grams of methanol with 10 grams of potassium hydroxide in a covered glass beaker. Mixing of the solid hydroxide was facilitated by adding a Teflon coated magnet and placing the beaker on a stirrer hot plate. Once the mixture was dissolved 120 grams of the solution was transferred to 1000 grams of flax oil. This mixture was agitated for 1 hour at room temperature using a Teflon coated bar magnet on a stirrer hotplate. After 1 hour the contents of the reaction vessel were transferred to a 2 liter glass separatory funnel and allowed to separate for 4 hours. After 4 hours the lower layer containing primarily glycerin was drained and set aside the upper layer was returned to a beaker for a second stage of reaction.

The second stage of reaction was accomplished by adding the remaining catalyst alcohol solution (90 g) to the safflower oil and agitating with a Teflon stirring bar as described above for 1 hour. The reaction contents were transferred to a 2 liter glass separatory funnel and allowed to separate overnight. After settling the lower layer containing glycerin, potassium hydroxide and alcohol was removed. The upper layer was placed on a rotary evaporator to substantially remove all remaining methanol. After the alcohol was removed the methyl ester was filtered on a glass fiber filter to remove residual glycerol catalyst and soaps. The residual material was used as a flax oil methyl ester substrate in further reactions.

### Example 11

### Isomerization of Safflower Methyl Ester with Peg 300 Potassium Alkylate

One hundred grams of safflower methyl ester (prepared according to example 8) was added to 13 g of PEG potassium alkylate (prepared according to example 1) in a round bottom flask. A Teflon coated stirring bar was added to the flask to afford agitation. The flask was placed in a constant temperature bath and stirred while it was held at 110° C. Vacuum (27″) was applied to the flask through a condenser. The reaction mixture bubbled vigorously for the first minutes due to the release of methanol. After two hours the vacuum was released and a sample of the reaction mixture was taken and added to deuterated chloroform in an NMR sample tube. The 400 MHz NMR spectrum was recorded. It was found that the methylene interrupt signal normally found at 2.78 ppm was greatly diminished and that new signals attributable to conjugated lipids had appeared at 6.25, 5.90, and 5.60 ppm.

### Example 12

### Isomerization Safflower Ethyl Esters with Peg 300 Potassium Alkylate Prepared from Aqueous Potassium Hydroxide and Peg 300

One hundred grams of safflower ethyl ester (prepared according to example 9) was added to 13 g of PEG potassium alkylate (prepared according to example 2) in a round bottom flask. A Teflon coated stirring bar was added to the flask to afford agitation. The flask was placed in a constant temperature bath and stirred while it was held at 110° C. Vacuum (27″) was applied to the flask through a condenser. The reaction mixture bubbled vigorously for the first minutes due to the release of methanol. After two hours the vacuum was released and a sample of the reaction mixture was taken and added to deuterated chloroform in an NMR sample tube. The 400 MHz NMR spectrum was recorded. It was found that the methylene interrupt signal normally found at 2.78 ppm was greatly diminished and that new signals attributable to conjugated lipids had appeared at 6.25, 5.90, and 5.60 ppm.

### Example 13

### No Isomerization of Safflower Ethyl Esters with Peg 300 and Potassium Carbonates

One hundred grams of safflower ethyl ester (prepared according to example 9) was added to 13 g of PEG potassium carbonate in a round bottom flask. A Teflon coated stirring bar was added to the flask to afford agitation. The flask was placed in a constant temperature bath and stirred while it was held at 110° C. Vacuum (27″) was applied to the flask through a condenser. The reaction mixture bubbled vigorously for the first minutes due to the release of methanol. After two hours the vacuum was released and a sample of the reaction mixture was taken and added to deuterated chloroform in an NMR sample tube. The 400 MHz NMR spectrum was recorded. It was found that the methylene interrupt signal normally found at 2.78 ppm was not altered by the treatment. This is consistent with the observation that no PEG alkylate catalyst formed using the metal carbonate as a source of base.

### Example 14

### Isomerization Safflower Ethyl Esters with Peg 300 Potassium Alkylate Prepared from Potassium Ethoxide and Peg 300

One hundred grams of safflower ethyl ester (prepared according to example 9) was added to 13 g of PEG potassium alkylate (prepared according to example 4) in a round bottom flask. A Teflon coated stirring bar was added to the flask to afford agitation. The flask was placed in a constant temperature bath and stirred while it was held at 110° C. Vacuum (27″) was applied to the flask through a condenser. The reaction mixture bubbled vigorously for the first minutes due to the release of methanol. After two hours the vacuum was released and a sample of the reaction mixture was taken and added to deuterated chloroform in an NMR sample tube. The 400 MHz NMR spectrum was recorded. It was found that the methylene interrupt signal normally found at 2.78 ppm was greatly diminished and that new signals attributable to conjugated lipids had appeared at 6.25, 5.90, and 5.60 ppm.

### Example 15

### Isomerization Safflower Ethyl Esters with Peg 300 Potassium Alkylate Prepared from Potassium Metal and Peg 300

One hundred grams of safflower ethyl ester (prepared according to example 9) was added to 13 g of PEG potassium alkylate (prepared according to example 4) in a round bottom flask. A Teflon coated stirring bar was added to the flask to afford agitation. The flask was placed in a constant temperature bath and stirred while it was held at 110° C. Vacuum (27″) was applied to the flask through a condenser. The reaction mixture bubbled vigorously for the first minutes due to the release of methanol. After two hours the vacuum was released and a sample of the reaction mixture was taken and added to deuterated chloroform in an NMR sample tube. The 400 MHz NMR spectrum was recorded. It was found that the methylene interrupt signal normally found at 2.78 ppm was greatly diminished and that new signals attributable to conjugated lipids had appeared at 6.25, 5.90, and 5.60 ppm.

### Example 16

### Isomerization Safflower Ethyl Esters with Peg 300 Cesium Alkylate Prepared from Cesium Hydroxide Monohydrate and Peg 300

Twenty five grams of safflower ethyl ester (prepared according to example 9) was added to 13 g of PEG cesium alkylate (prepared according to example 1) in a round bottom flask. A Teflon coated stirring bar was added to the flask to afford agitation. The flask was placed in a constant temperature bath and stirred while it was held at 110° C. Vacuum (27″) was applied to the flask through a condenser. The reaction mixture bubbled vigorously for the first minutes due to the release of methanol. After two hours the vacuum was released and a sample of the reaction mixture was taken and added to deuterated chloroform in an NMR sample tube. The 400 MHz NMR spectrum was recorded. It was found that the methylene interrupt signal normally found at 2.78 ppm was greatly diminished and that new signals attributable to conjugated lipids had appeared at 6.25, 5.90, and 5.60 ppm.

### Example 17

### Isomerization Safflower Ethyl Esters with Peg 300 Rubidium Alkylate Prepared from Rubidium Hydroxide Solution and Peg 300

Twenty five grams of safflower ethyl ester (prepared according to example 9) was added to 3.25 g of PEG rubidium alkylate (prepared according to example 1) in a round bottom flask. A Teflon coated stirring bar was added to the flask to afford agitation. The flask was placed in a constant temperature bath and stirred while it was held at 110° C. Vacuum (27″) was applied to the flask through a condenser. The reaction mixture bubbled vigorously for the first minutes due to the release of methanol. After two hours the vacuum was released and a sample of the reaction mixture was taken and added to deuterated chloroform in an NMR sample tube. The 400 MHz NMR spectrum was recorded. It was found that the methylene interrupt signal normally found at 2.78 ppm had disappeared and that new signals attributable to conjugated lipids had appeared at 6.25, 5.90, and 5.60 ppm.

### Example 18

### Isomerization of Flax Methyl Ester with Peg 300 Potassium Alkylate

One hundred grams of flax methyl ester (prepared according to example 8) was added to 13 g of PEG potassium alkylate (prepared according to example 1) in a round bottom flask. A Teflon coated stirring bar was added to the flask to afford agitation. The flask was placed in a constant temperature bath and stirred while it was held at 110° C. Vacuum (27″) was applied to the flask through a condenser. The reaction mixture bubbled vigorously for the first minutes due to the release of methanol. After two hours the vacuum was released and a sample of the reaction mixture was taken and added to deuterated chloroform in an NMR sample tube. The 400 MHz NMR spectrum was recorded. It was found that the methylene interrupt signal normally found at 2.78 ppm was greatly diminished and that a complex pattern of new signals attributable to conjugated lipids had appeared between 5.5 and 6.5 ppm.

### Example 19

### Isomerization Safflower Ethyl Esters with Peg 300 Tetramethyl Ammonium Alkylate Prepared from Tetramethylammonium Hydroxide Solution and Peg 300

Tetramethyl ammonia hydroxide (488 mg) and PEG 300 (3.0 g) were mixed in a round bottom flask under vacuum at 110° C. for 2 hours. Twenty five grams of safflower ethyl ester (prepared according to example 9) was added to 3.25 g of the PEG tetramethyammonium alkylate in the flask. A Teflon coated stirring bar was added to the flask to afford agitation. The flask was placed in a constant temperature bath and stirred while it was held at 110° C. Vacuum (27″) was applied to the flask through a condenser. The reaction mixture bubbled vigorously for the first minutes due to the release of methanol. After two hours the vacuum was released and a sample of the reaction mixture was taken and added to deuterated chloroform in an NMR sample tube. The 400 MHz NMR spectrum was recorded. It was found that the methylene interrupt signal normally found at 2.78 ppm had disappeared and that new signals attributable to conjugated lipids had appeared at 6.25, 5.90, and 5.60 ppm.

### Example 20

### Isomerization of Safflower Methyl Ester with Polypropylene Glycol (Arcol® Polyol PPG 425) Potassium Alkylate

One hundred grams of safflower methyl ester (prepared according to example 8) was added to 13 g of polypropylene glycol potassium alkylate (prepared according to example 7) in a round bottom flask. A Teflon coated stirring bar was added to the flask to afford agitation. The flask was placed in a constant temperature bath and stirred while it was held at 110° C. Vacuum (27″) was applied to the flask through a condenser. The reaction mixture bubbled vigorously for the first minutes due to the release of methanol. After two hours the vacuum was released and a sample of the reaction mixture was taken and added to deuterated chloroform in an NMR sample tube. The 400 MHz NMR spectrum was recorded. It was found that the methylene interrupt signal normally found at 2.78 ppm was greatly diminished and that new signals attributable to conjugated lipids had appeared at 6.25, 5.90, and 5.60 ppm.

What is claimed is:

1. A process for producing a polyethylene alkylate catalyst comprising reacting an alkali base, selected from the group consisting of hydroxide, alkoxide, metal and hydride, with a polyether alcohol solvent, under vacuum at a temperature in the range of 100° C.-150° C., so as to produce a non volatile, non toxic polyether alkylate catalyst.

2. A strong base catalyst composition comprising a non volatile, non toxic polyether alkylate produced by reaction between an alkali base, selected from the group consisting of hydroxide, alkoxide, metal and hydride, and a polyether alcohol.

3. A process for producing an isomeric conjugated linoleic acid (CLA)-rich alkyl ester mixture comprising reacting a linoleic acid-rich oil reactant in the presence of a catalytic amount of a strong base comprising a non volatile non toxic polyether alkylate at a temperature above 50° C. and separating said CLA-rich alkyl ester mixture.

4. A catalyst according to claim 2 where the polyether alcohol is selected from the group consisting of a polymer of ethylene glycol, a polymer of propylene glycol, an alkyl ether of an alkanol and a polymer of ethylene glycol, an alkyl ether of an alkanol and a polymer of propylene glycol, and a copolymer of ethylene glycol and propylene glycol.

5. A catalyst according to claim 2 wherein the alkali base is selected from solids and solutions of a Group 1 metal, metal hydroxide, metal alkoxide, metal carbonate, and a metal carbonate.

6. A process according to claim 3 where the reactant is an ester rich in linoleic acid and the product is an ester of conjugated linoleic acid.

7. A process according to claim 3 where the reactant is selected from the group consisting of a linoleic rich ester derived from at least one of safflower, sunflower and solin oil

8. A process according to claim 3 where the reactant is selected from the group consisting of a methyl ester, an ethyl ester, and an alkyl ester rich in linoleic acid.

9. A process according to claim 8 where the linoleic rich alkyl ester is derived from the group consisting of flax and -perilla oil.

10. A process according to claim 8 where the alkyl ester is selected from the group consisting of methyl ester and ethyl ester.

11. A process according to claim 1 wherein said solvent includes a co-solvent.

12. A process according to claim 11 where the co-solvent is selected from the group consisting of dimethylsulfoxide, N-methylpyrrolidone and polyether alcohol.
